SEQUENCE LISTING (8 sequences)

SEQ ID NO: 1 - protein, 23 aa, Artificial Sequence
  Description of Artificial Sequence Synthetic peptide
  MOD_RES (23)..(23): Lys(DABCYL)
GEIAALERENAALEWEIAALEQK

SEQ ID NO: 2 - protein, 42 aa, Artificial Sequence
  Description of Artificial Sequence Synthetic polypeptide
  MOD_RES (42)..(42): Lys(FITC)
CFNKTFLDKFNHEAEDLFYQSSLARIAALKYKNAALKKKIAK

SEQ ID NO: 3 - protein, 38 aa, Artificial Sequence
  Description of Artificial Sequence Synthetic polypeptide
  MOD_RES (38)..(38): Lys(FITC)
CFNKTFLDKFNHEAEDLFYQSSLARIAALKYKNAALKK

SEQ ID NO: 4 - protein, 46 aa, Artificial Sequence
  Description of Artificial Sequence Synthetic polypeptide
  MOD_RES (46)..(46): Lys(FITC)
CFNKTFLDKFNHEAEDLFYQSSLARIAALKYKNAALKKKIAALKQK

SEQ ID NO: 5 - protein, 65 aa, Artificial Sequence
  Description of Artificial Sequence Synthetic polypeptide
  MOD_RES (1)..(1): Lys(DABSYL)
  MOD_RES (65)..(65): Lys(FITC)
KQELAAIEWELAANERELAAIEGCFNKTFLDKFNHEAEDLFYQSSLARIAALKYKNAALKKKIAK

SEQ ID NO: 6 - protein, 61 aa, Artificial Sequence
  Description of Artificial Sequence Synthetic polypeptide
  MOD_RES (1)..(1): Lys(DABSYL)
  MOD_RES (61)..(61): Lys(FITC)
KQELAAIEWELAANERELAAIEGCFNKTFLDKFNHEAEDLFYQSSLARIAALKYKNAALKK

SEQ ID NO: 7 - protein, 69 aa, Artificial Sequence
  Description of Artificial Sequence Synthetic polypeptide
  MOD_RES (1)..(1): Lys(DABSYL)
  MOD_RES (69)..(69): Lys(FITC)
KQELAAIEWELAANERELAAIEGCFNKTFLDKFNHEAEDLFYQSSLARIAALKYKNAALKKKIAALKQK

SEQ ID NO: 8 - protein, 24 aa, Artificial Sequence
  Description of Artificial Sequence Synthetic peptide
CFNKTFLDKFNHEAEDLFYQSSLA

(19) United States
(12) Patent Application Publication
(10) Pub. No.: US 2023/0194525 A1
(43) Pub. Date: Jun. 22, 2023

(54) PEPTIDE BASED PROBES FOR THE DETECTION OF SARS-COV-2

(71) Applicant: Massachusetts Institute of Technology, Cambridge, MA (US)

(72) Inventors: Pranam CHATTERJEE, Cambridge, MA (US); Raghava Manvitha PONNAPATI, Cambridge, MA (US); Soumya Pratap TRIPATHY, Cambridge, MA (US); Joseph M. JACOBSON, Newton, MA (US)

(21) Appl. No.: 17/661,428

(22) Filed: Apr. 29, 2022

Related U.S. Application Data
(60) Provisional application No. 63/182,537, filed on Apr. 30, 2021.

(51) Int. Cl. G01N 33/569

(52) U.S. Cl. CPC G01N 33/56983; G01N 2333/165; G01N 2469/10

(57) ABSTRACT

A highly specific molecular diagnostic for the detection of an intended target protein within a matter of minutes employing a peptide beacon, the peptide beacon having a stem section having two ends attached to a fluorophore and a quencher and a loop section having a receptor sequence for binding with the intended target protein, the two ends forming a coiled-coil structure when the receptor sequence is unbound with the intended target protein and an open-coil structure when the receptor sequence is bound with the intended target protein, wherein the peptide beacons are able to provide a signal for the detection of the receptor binding domain of the intended target protein, such as SARS-CoV-2 spike protein, by the stem section transitioning from the coiled-coil structure to the open-coil structure that moves the fluorophore away from the quencher, resulting in an increase in the fluorescence yield of the peptide beacon.

### RELATED APPLICATIONS

The present application claims priority to U.S. Provisional Application Ser. No. 63/182,537 filed Apr. 30, 2021 and entitled “PEPTIDE BASED PROBES FOR THE DETECTION OF SARS-COV-2”, which is hereby incorporated by reference in its entirety.

The instant application contains a Sequence Listing which has been submitted electronically in ASCII format and is hereby incorporated by reference in its entirety. Said ASCII copy, created on Dec. 20, 2022, is named 5301_04US02_SL.txt and is 5,254 bytes in size.

### FIELD OF TECHNOLOGY

The present disclosure relates to viral detection, and more particularly relates to detection of the virus SARS-CoV-2 by use of a highly specific molecular diagnostic comprising a peptide beacon.

### BACKGROUND

Over the past year, SARS-CoV-2 has emerged as a highly pathogenic coronavirus and has nowspread to over 200 countries, infecting over 50 million people worldwide and killing over 1 million people as of October 2020. Economies have crashed, travel restrictions have been imposed, and public gatherings have been canceled, all while a sizeable portion of the human population remains quarantined. Rapid transmission dynamics as well as a wide range of symptoms, from a simple dry cough to pneumonia and death, are common characteristics of coronavirus disease 2019 (COVID-19) [Wu, J. T. et al. Estimating clinical severity of COVID-19 from the transmission dynamics in Wuhan, China. Nat. Med. 26, 506-510 (2020)]. With no cures readily available [Lurie, N., Saville, M., Hatchett, R. & Halton, J. Developing covid-19 vaccines at pandemic speed. N. Engl. J. Med. 382, 1969-1973 (2020)], and only limited vaccine availability, there is a pressing need for fast and effective detection of the virus.

Existing viral detection methods rely on complex, multistep processes, such as PCR, LAMP, or CRISPR-based method for sufficiently sensitive detection. These methods are generally quite costly and require a long duration to yield a result.

Thus, there exists a present need in the art for a rapid, point-of-care, viral detection assay, that is specific to SARS-CoV-2 and provides accurate results.

### SUMMARY

The present disclosure relates to a rapid, sensitive and highly specific molecular diagnostic comprising a peptide beacon. The peptide beacons of the present disclosure can be used for the detection of any intended target protein. Such a highly specific molecular diagnostic employing peptide beacons of the present disclosure are critical to facilitate human economic and societal activity in the presence of the current SARS-CoV-2 or future pandemic. In order to be optimally impactful, the diagnostic can provide point-of-care and be able to detect target proteins, such as SARS-CoV-2 or other target virus within a matter of minutes.

In some aspects, the assembled peptide beacon comprises a stem having two ends and a loop proximately located between the two stem ends. A fluorophore-quencher pair can be attached to the two ends of the stem. The two stem ends configured to coil over each other to create a coiled-coil structure and the loop comprises a receptor sequence for the intended target protein. The receptor sequence being capable of binding to the intended target protein and transitioning the fluorescence state of the peptide beacon from a low-fluorescence state to a high-fluorescence state. In some aspects, the low-fluorescence state occurs when a distance between the quencher (Q) and the fluorophore (F) is approximately the Förster distance. In some aspects, the high-fluorescence state occurs when the distance between the quencher (Q) and the fluorophore (F) is greater than the Förster distance.

In some aspects, the present disclosure relates to computationally designed and developed peptide beacons.

In some aspects, molecular beacons may be oligonucleotides or peptide sequences with a first modified end having an attached quencher and a second modified end having an attached fluorophore. In some aspects, molecular beacons use binding-specific conformational changes to produce a detectable signal. In the absence of the target molecule, the terminal ends of the molecular beacon are in close proximity to each other bringing the fluorophore/quencher pair in proximity and thereby minimizing fluorescence emission. Hybridization of the target molecule to the targeting portion in the middle of the beacon causes a conformational change that separates the fluorophore/quencher pair resulting in an increase in fluorescence emission.

In some aspects, the peptide beacons are based on a novel SARS-CoV-2 spike protein binding peptide.

In some aspects, the peptide beacons are able to detect a receptor binding domain (RBD) of the SARS-CoV-2 spike protein. In some aspects, the with a peptide beacons are able to detect a RBD of the SARS-CoV-2 spike protein with a limit of detection (LOD) of about 50 to about 60 pM and 10-fold fluorescence signal than the background within 10 minutes of turn-around time, in some aspects less than 10 minutes of turn-around time.

In some aspects, the peptide beacons are integrated with on-chip optical sensors to construct a point-of care antigen test platform, such as for SARS-CoV-2.

In some aspects, the peptide beacon comprises a peptide sequence having the sequence identified as SEQID No. 1.

In some aspects, the peptide beacon comprises a peptide sequence having the sequence identified as SEQID No. 2.

In some aspects the peptide beacon comprises a peptide sequence having the sequence identified as SEQID No. 3.

In some aspects, the peptide beacon is synthesized from a peptide sequencing having the sequence identified as SEQID No. 4.

In some aspects, the synthesized peptide beacon has the sequence identified as SEQID No. 5.

In some aspects, the synthesized peptide beacon has the sequence identified as SEQID No. 6.

In some aspects, the synthesized peptide beacon has the sequence identified as SEQID No. 7.

In some aspects, the peptide beacon is synthesized from a peptide sequencing having the sequence identified as SEQID No. 1 conjugated with the sequence identified as SEQID No. 2.

In some aspects, the peptide beacon is synthesized from a peptide sequencing having the sequence identified as SEQID No. 1 conjugated with the sequence identified as SEQID No. 3.

In some aspects, the peptide beacon is synthesized from a peptide sequencing having the sequence identified as SEQID No. 1 conjugated with the sequence identified as SEQID No. 4.

In some aspects, the peptide beacon is configured to have a stem section and a loop section, the stem section comprising a coiled-coil peptide and the loop section comprising a receptor for the intended target protein.

In some aspects, the loop section of the peptide beacon comprises a sequence listing comprising the sequence identified as SEQID No. 8.

In some aspects, the peptide beacon comprises a fluorophore-quencher pair attached to the two ends of the stem section.

In some aspects, the peptide beacon is configured such that when the target binds to the loop section, the stem section opens up moving the fluorophore away from the quencher, resulting in an increase in the fluorescence yield of the system. In some aspects, the system is configured to sense an increase in fluorescence yield.

### BRIEF DESCRIPTION OF THE DRAWINGS

Other aspects, advantages and novel features of the invention will become more apparent from the following detailed description of the invention when considered in conjunction with the accompanying drawing, wherein:

FIG. 1 is an example synthesis of a peptide beacon, according to certain embodiments of the present disclosure.

FIG. 2 is an overview of an example method for designing coiled-coil peptide beacons for an intended target protein, according to certain embodiments of the present disclosure.

FIG. 3 is an overview of an example generative model for designing a binding loop of a peptide beacon for an intended target protein, according to certain embodiments of the present disclosure.

FIG. 4A is a graph illustrating the decrease in fluorescence intensity relating to the synthesis of three peptide beacons (RL1, RL2 and RL3), according to certain embodiments of the present disclosure, demonstrated according to an example.

FIG. 4B is a graph illustrating the titration between portion R and portion L of each of the three peptide beacons (RL1, RL2 and RL3) of FIG. 4A relating to the synthesis of various peptide beacons, according to certain embodiments of the present disclosure, demonstrated according to an example.

FIG. 4C is a photograph of an SDS-PAGE gel showing bands for each of the three peptide beacons (RL1, RL2 and RL3) of FIG. 4A and the three different peptides (L1, L2 and L3) that conjugate with peptide (R) to form the three different peptide beacons (RL1, RL2 and RL3) relating to the synthesis of various peptide beacons, according to certain embodiments of the present disclosure, demonstrated according to an example.

FIG. 4D is a MALDI-TOF mass spectrum of the conjugated peptide beacon R+L1 showing peaks corresponding to R, L1 and RL1, according to certain embodiments of the present disclosure, demonstrated according to an example.

FIG. 4E is a MALDI-TOF mass spectrum of the conjugated peptide beacon R+L2 showing peaks corresponding to R, L2 and RL2, according to certain embodiments of the present disclosure, demonstrated according to an example.

FIG. 4F is a MALDI-TOF mass spectrum of the conjugated peptide beacon R+L3 showing peaks corresponding to R, L3 and RL3, according to certain embodiments of the present disclosure, demonstrated according to an example.

FIG. 5A is a graph of an HPLC chromatogram of the conjugated peptide beacon R+L1 plotted with an HPLC chromatograph of R and L1, according to certain embodiments of the present disclosure, demonstrated according to an example.

FIG. 5B is a graph of an HPLC chromatogram of the conjugated peptide beacon R+L2 plotted with an HPLC chromatograph of R and L2, according to certain embodiments of the present disclosure, demonstrated according to an example.

FIG. 5C is a graph of an HPLC chromatogram of the conjugated peptide beacon R+L3 plotted with an HPLC chromatograph of R and L3, according to certain embodiments of the present disclosure, demonstrated according to an example.

FIG. 5D is a MALDI-TOF mass spectrum of the conjugated peptide beacon R+L1 showing peaks corresponding to R, L1 and RL1, according to certain embodiments of the present disclosure, demonstrated according to an example.

FIG. 5E is a MALDI-TOF mass spectrum of the conjugated peptide beacon R+L2 showing peaks corresponding to R, L2 and RL2, according to certain embodiments of the present disclosure, demonstrated according to an example.

FIG. 5F is a MALDI-TOF mass spectrum of the conjugated peptide beacon R+L3 showing peaks corresponding to R, L3 and RL3, according to certain embodiments of the present disclosure, demonstrated according to an example.

FIG. 6A is a schematic relating to the detection of a receptor binding domain (RBD) of the SARS-CoV-2 spike protein using peptide beacons, according to certain embodiments of the present disclosure, demonstrated according to another example, wherein the peptide beacon shown transitioning from a low-fluorescent state to a high-fluorescent state, the peptide beacon configured to comprise a stem portion and a loop portion, the stem portion comprising a coiled-coil peptide and the loop portion comprising a receptor for the intended protein, with a fluorophore-quencher pair attached to the two ends of the stem portion, the two ends of the stem portion in a Forster distance relating to the non-fluorescent state and greater than the Forster distance relating to the fluorescent state when the target binds to the loop portion causing the coiled-coil stem portion to open up and move the fluorophore away from the quencher, which causes an increase in the fluorescence yield of the system.

FIG. 6B is a graph relating to the detection of a receptor binding domain (RBD) of the SARS-CoV-2 spike protein using the titration of the target recombinant RBD of the SARS-CoV-2 spike protein with the three peptide beacons (RL1, RL2 and RL3), according to certain embodiments of the present disclosure, demonstrated according to another example.

FIG. 6C is a graph relating to the detection of a receptor binding domain (RBD) of the SARS-CoV-2 spike protein using the titration of the target recombinant RBD of the SARS-CoV-2 spike protein and Influenza A-HA-H1 with peptide beacon RL1, according to certain embodiments of the present disclosure, demonstrated according to another example.

FIG. 6D is a graph relating to the detection of a receptor binding domain (RBD) of the SARS-CoV-2 spike protein using the titration of the target recombinant RBD of the SARS-CoV-2 spike protein and Influenza A-HA-H1 with peptide beacon RL2, according to certain embodiments of the present disclosure, demonstrated according to another example.

FIG. 6E is a graph relating to the detection of a receptor binding domain (RBD) of the SARS-CoV-2 spike protein using the titration of the target recombinant RBD of the SARS-CoV-2 spike protein and Influenza A-HA-H1 with peptide beacon RL3, according to certain embodiments of the present disclosure, demonstrated according to another example.

### DETAILED DESCRIPTION

Disclosed herein are peptide beacons that can be employed for one-step detection of viruses, such as SARS-CoV-2. The disclosed detection employing the peptide beacons is highly sensitive and provides effective detection of a target protein in a short amount of time.

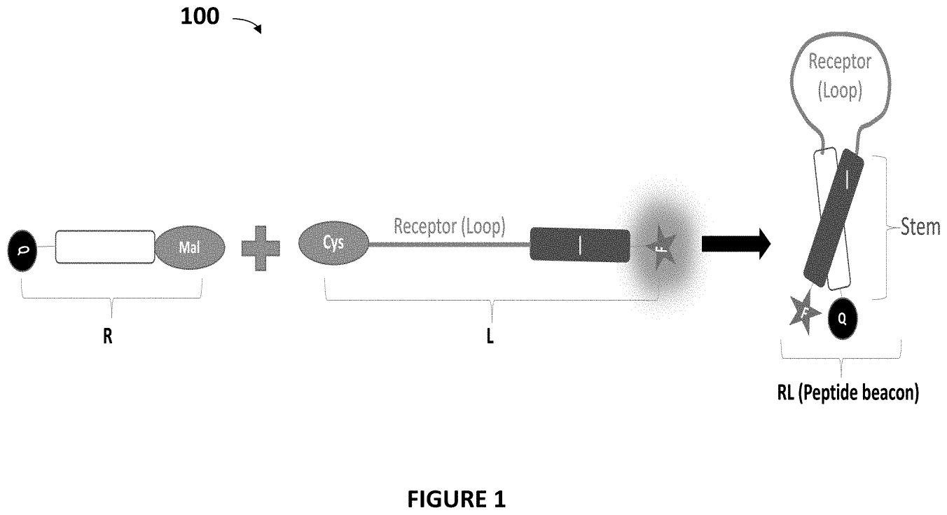

Referring now to FIG. 1, an example synthesis 100 of a peptide beacon according to certain embodiments of the present disclosure is shown. Each peptide beacon generally comprises peptide R conjugated with peptide L to synthesize the peptide beacon RL. In some aspects, peptide R can be conjugated with peptide L through the use of coupling chemistry. In some preferred aspects, peptide R can be conjugated with peptide L through the use of a maleimide linker, as shown in example synthesis 100.

In some preferred aspects, peptide R comprises a coupling linker and a quencher (Q). In some aspects, the coupling linker is present at the N-terminus of peptide R and is capable of coupling with an amino acid present at an N-terminus of peptide L to conjugate peptide R with peptide L to form peptide beacon RL.

In some preferred aspects, the coupling linker is a maleimide linker proximately located at the N-terminus of peptide R. In some preferred aspects, the maleimide linker present at the N-terminus of peptide R is capable of coupling with a cysteine amino acid present at an N-terminus of L to conjugate peptide R with peptide L to form peptide beacon RL.

In some aspects, the quencher (Q) is proximately located the C-terminus of peptide R. In some preferred aspects, the quencher (Q) of peptide R comprises an in-sequence lysine amino acid labeling by DABCYL.

In aspects, peptide L comprises a fluorophore (F) and a receptor sequence for the intended target protein. In some preferred aspects, the fluorophore (F) is proximately located the C-terminus of peptide L. In some preferred aspects, the fluorophore is fluorescein isothiocyanate (FITC). In some preferred aspects, the N-terminus of peptide L is a cysteine amino acid. In some preferred aspects, the receptor sequence comprises a portion of peptide L.

In some alternative aspects, peptide R comprises a coupling linker and a fluorophore (F) and peptide L comprises the quencher (Q) and the receptor sequence. In still some other alternative aspects, the coupling linker can be located on either peptide R or peptide L to form peptide beacon RL.

The fluorescence of a fluorophore, such as fluorescein isothiocyanate (FITC) in example synthesis 100, in peptide L decreases after peptide L conjugates with peptide R to form peptide beacon RL due to the proximity of the fluorophore with a quencher in peptide R, such as DABCYL in example synthesis 100. Thus, the decrease in fluorescence over time after peptide R is added to peptide L can provide an indication of synthesis of the peptide beacon, such as RL shown in synthesis 100.

In some embodiments, gel electrophoresis, mass spectrometry, or any other appropriate analysis may also be used to confirm synthesis of peptide beacons from conjugation between peptide R and peptide L.

The present disclosure includes using peptide sequences, for assembling peptide beacons, according to certain embodiments of the present invention. In some preferred aspects, peptide R has SEQID NO. 1, as provided in Table 1. In some preferred aspects, peptide L has SEQID NO. 2, SEQID NO. 3, or SEQID NO. 4, as provided in Table 1.

TABLE 1
Peptide Subunits for synthesis of
peptide beacons

| Peptide | |
|---|---|
| (SEQ ID No.) | SEQUENCE |
| R | (Mal)GEIAALERENAALEWE |
| (SEQ ID No. 1) | IAALEQ{Lys(DABCYL)} |
| L1 | CFNKTFLDKFNHEAEDLFYQS |
| (SEQ ID No. 2) | SLARIAALKYKNAALKKKIA |
| | {LYS(FITC)} |
| L2 | CFNKTFLDKFNHEAEDLFYQS |
| (SEQ ID No. 3) | SLARIAALKYKNAALK{LYS |
| | (FITC)} |
| L3 | CFNKTFLDKFNHEAEDLFYQSS |
| (SEQ ID No. 4) | LARIAALKYKNAALKKKIAALK |
| | Q{LYS(FITC)} |

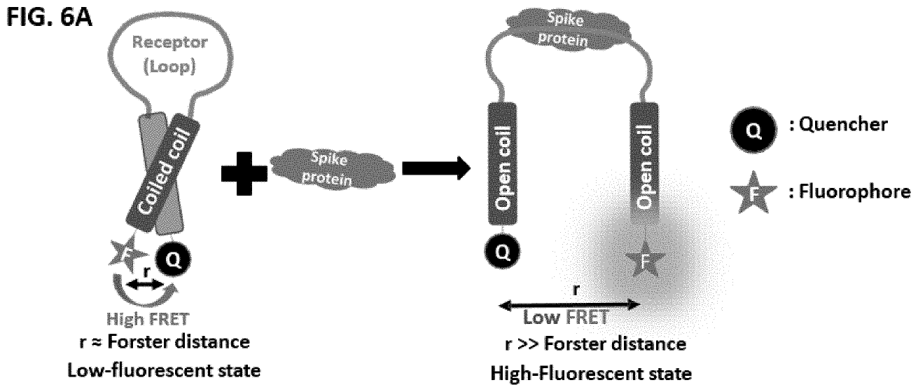

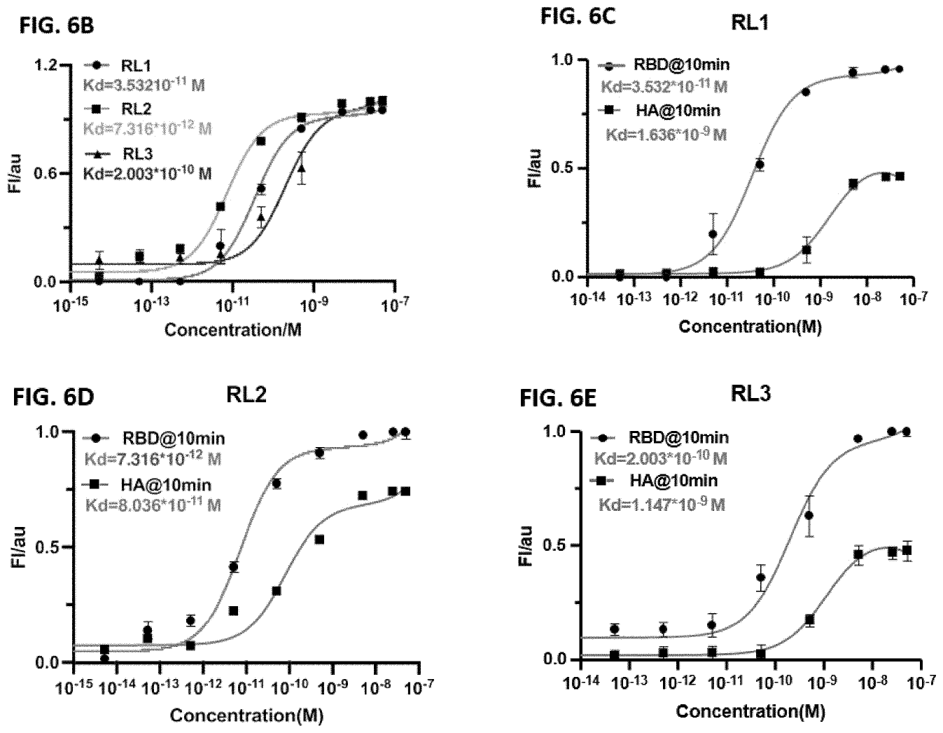

The present disclosure includes peptide beacons assembled using peptide subunit sequences according to certain embodiments of the present invention. In some aspects, the assembled peptide beacon comprises a stem having two ends and a loop proximately located between the two stem ends. The two stem ends are configured to coil over each other to create a coiled-coil structure and the loop comprises a receptor sequence for the intended target protein. In some aspects, the stem of the assembled peptide beacon comprises peptide subunit R. In some aspects, the stem of the assembled peptide beacon comprises a first portion of peptide subunit L and the loop of the assembled peptide beacon comprises a second portion of peptide subunit L. In some aspects, the stem comprises peptide subunit R and a first portion of peptide subunit L, which are coiled over each other to create a coiled-coil structure, and the loop of the assembled peptide beacon comprises a second portion of peptide subunit L that forms the receptor sequence for the intended target protein. The receptor sequence being capable of binding to the intended target protein and transitioning the fluorescence state of the peptide beacon from a low-fluorescence state to a high-fluorescence state, such as shown in FIG. 6A. In some aspects, the low-fluorescence state occurs when a distance between the quencher (Q) and the fluorophore (F) is approximately the Förster distance. In some aspects, the high-fluorescence state occurs when the distance between the quencher (Q) and the fluorophore (F) is greater than the Förster distance.

In some preferred aspects, peptide RL1 has SEQID No. 1 as peptide subunit R and SEQID No. 2 as peptide subunit L. In some preferred aspects, peptide RL2 has SEQID No. 1 as peptide subunit R and SEQID No. 3 as peptide subunit L. In some preferred aspects, peptide RL3 has SEQID No. 1 as peptide subunit R and SEQID No. 4 as peptide subunit L.

In some preferred aspects, peptide beacon RL1 has SEQID NO. 5, peptide beacon RL2 has SEQID NO. 6, and peptide beacon RL3 has SEQID NO. 7, as provided in Table 2.

TABLE 2
Peptide sequences of peptide beacons

| PEPTIDE | |
|---|---|
| BEACON | |
| (SEQ ID NO.) | SEQUENCE |
| RL1 | {Lys(DABSYL)}QELAAIEW |
| (SEQ ID NO 5.) | ELAANERELAAIEGCFNKTFL |
| | DKFNHEAEDLFYQSSLARIAA |
| | LKYKNAALKKKIA{LYS |
| | (FITC)} |
| RL2 | {Lys(DABSYL)}QELAAIEWE |
| (SEQ ID NO. 6) | LAANERELAAIEGCFNKTFLDK |
| | FNHEAEDLFYQSSLARIAALKY |
| | KNAALK{LYS(FITC)} |
| RL3 | {Lys(DABSYL)}QELAAIEWE |
| (SEQ ID NO. 7) | LAANERELAAIEGCFNKTFLDK |
| | FNHEAEDLFYQSSLARIAALKY |
| | KNAALKKKIAALKQ |
| | {LYS(FITC)} |

In some aspects, the stem of the assembled peptide beacon comprises peptide subunit R having SEQID No. 1 and a first portion of peptide subunit L1, and the loop of the assembled peptide beacon comprises a second portion of peptide subunit L1, which is the receptor sequence for the intended target protein. In some aspects, the stem of the assembled peptide beacon comprises peptide subunit R having SEQID No. 1 and a first portion of peptide subunit L2, and the loop of the assembled peptide beacon comprises a second portion of peptide subunit L2, which is the receptor sequence for the intended target protein. In some aspects, the stem of the assembled peptide beacon comprises peptide subunit R having SEQID No. 1 and a first portion of peptide subunit L3, and the loop of the assembled peptide beacon comprises a second portion of peptide subunit L3, which is the receptor sequence for the intended target protein.

In some preferred aspects, the second portion of peptide subunit L that is the receptor sequence comprises SEQID No. 8, shown in Table 3. The receptor sequence being capable of binding to the intended target protein and transitioning the fluorescence state of the peptide beacon from a low-fluorescence state to a high-fluorescence state.

TABLE 3
Receptor sequence of peptide beacons

| | |
|---|---|
| Receptor | CFNKTFLDKFNH |
| Sequence | EAEDLFYQSSLA |
| (SEQ ID NO. 8) | |

As discussed in more detail in relation to the examples below, a peptide beacon according to the present disclosure will assume a closed configuration having the coiled-coil structure when no target is bound. When a target protein, such as a viral spike protein, is bound by the receptor loop formed by the beacon in the closed formation, the beacon undergoes a conformational shift and the quencher and the fluorophore move away from one another to an open configuration having an open-coil structure, and the fluorophore will fluoresce. The transition from the low-fluorescent state to the high-fluorescent state, such as shown in FIG. 6A, can be used to detect the presence of a specific molecule or analyte.

Peptide beacons according to the present disclosure may generally exhibit common features to support efficiency of manufacture and function. The structure of the peptide beacon generally comprises a stem and a loop. The sequence of peptide R comprises a sequence for the right arm (r) of the stem. The sequence of peptide L comprises a sequence for the receptor (loop) and a sequence for the left arm (1) of the stem. In the structure of a peptide beacon, the right arm (r) and the left arm (1) coil over each other to form a coiled-coil structure, which can serve as a stem in the peptide beacon. Peptide R can be conjugated with peptide L to synthesize a peptide beacon RL, which can be employed as a probe for a highly specific molecular diagnostic.

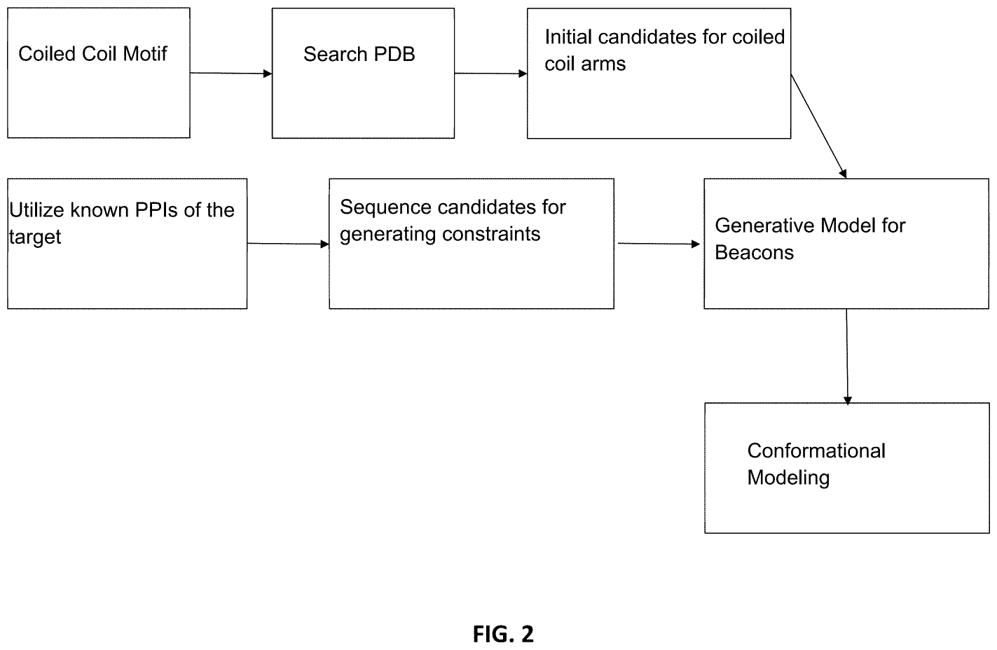

The following example machine learning method, shown in the flowchart of FIG. 2, can be used for selecting the coiled coil arms (r and 1) or the loop sequence of the peptide beacon.

The example method can be used for an initial design of coiled coil arms, e.g., r and 1 as discussed above. The right and left arm of the coiled coil beacons can be designed using starting coiled coil sequences obtained, such by searching the Protein Data Bank (PDB) for proteins with a coiled coil motif. In some cases, all proteins from a bank, such as PDB, may be retrieved. Using a protein-protein docking protocol, e.g., Rosetta Docking Protocol, the candidate sequences for the right and left arms can be docked against a target of interest and a binding strength score can be predicted. The docking can be evaluated using the docking protocol to identify top candidates for the right and left arms. The arms may also be tested against the target individually, such as by using a degradation assay in the lab. Designs for downstream processing for the left and right arms can be selected based on the lab results and docking models.

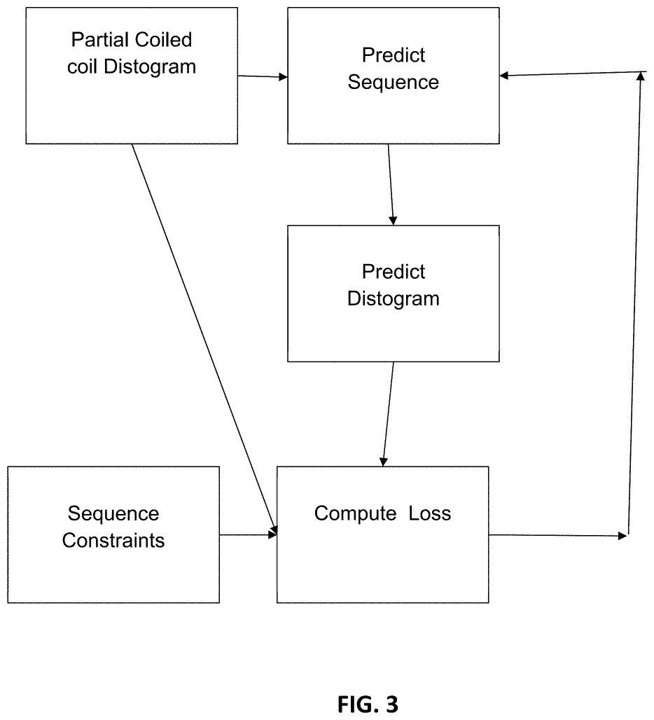

The example method shown in the flowchart of FIG. 3 can be used for designing a binding loop, according to the present disclosure. The binding loop can be configured to lay between the coiled coil arms and provide specific targeting of an intended protein for the peptide beacon.

A binding portion of the coiled coil beacon can be designed by using a distogram predicting machine learning model. In some preferred aspects, the distogram predicting model is a trRosetta distogram prediction model. A partial distogram with the coiled coil portions of the protein can be supplied to a generative model configured to predict an initial random sequence as a candidate for the binding loop. The sequence can be iteratively optimized, such as by using the distogram prediction model to predict the distogram of the complete protein formed by the initial random sequence with the coiled coil arms.

Various loss can be used to configure the distogram prediction. The loss used can a coiled coil motif loss, a generative loss, a loss based on sequence constraints, or a combination thereof. Sequence based constraints can be used to favor proteins that have binding affinity to the target of interest. The constraints can be selected based on a known binding partner to the target and then forcing a model to complete the distogram using a sequence, which in some preferred aspects has at least 70% similarity to the known binding partner. In some aspects, the similarity is least 50% similarity, in some aspects at least 55% similarity, in some aspects at least 60% similarity, in some aspects at least 65% similarity, in some aspects at least 70% similarity, in some aspects at least 75% similarity, in some aspects at least 80% similarity, in some aspects at least 85% similarity, in some aspects at least 90% similarity, in some aspects at least 95% similarity, to the known binding partner. In some aspects, one or more residues that play a role in the binding with the target of interest may be constrained in the generative model, allowing the generative model freedom to change or more other residues.

The example method can be used for improving the designs of coiled coil arms, such as for peptide beacons according to the present disclosure. Some or all parts of a coiled coil beacon can be combined together for additional stages of modeling. The coiled coil beacons can be further improved using conformational modeling with a binding loop in between the coiled coils and mutagenesis (computational and lab-based).

Since the 2003 SARS epidemic, it has been widely known that the angiotensin-converting enzyme 2 (ACE2) receptor is critical for SARS-CoV entry into host cells [Du, et al., “The spike protein of sars-cov—a target for vaccine and therapeutic development”, Nat Rev. Microbiol. (2009)]. ACE2 is a monocarboxypeptidase, widely known for cleaving various peptides within the renin—angiotensin system [Tipnes, et al., “A human homolog of angiotensin-converting enzyme: cloning and functional expression as a captopril-insensitive carboxypeptidase”, Journal of Biological Chemistry (2000)]. Functionally, there are two forms of ACE2. The full-length ACE2 contains a structural transmembrane domain, which anchors its extracellular domain to the plasma membrane [Du, et al., “The spike protein of sars-cov a target for vaccine and therapeutic development” Nat. Rev. Microbiol. (2009)]. The extracellular domain has been demonstrated as a receptor for the spike (S) protein of SARS-CoV, and recently, for the SARS-CoV-2.

In certain embodiments of the present invention, the peptide beacons are based on a novel SARS-CoV-2 spike protein binding peptide. In some embodiments, the peptide beacons are able to detect RBD of the SARS-CoV-2 spike protein with a LoD of 50-60 pM and 10-fold fluorescence signal than the background within 10 minutes of turn-around time.

In some embodiments, the peptide beacons are integrated with on-chip optical sensors to construct a point-of care antigen test platform, such as for SARS-CoV-2.

In some aspects, the peptide beacons can be employed for detection of a sample, such as a sample from a mammal, preferably a human. In some aspects, the sample can be a bodily fluid, such as blood, urine, saliva, nasal mucus, or the like. In some preferred aspects, the sample is a nasal fluid, nasopharyngeal fluid, oropharyngeal fluid, condensed breath or combination thereof.

### EXAMPLES

### Example 1: Confirming Synthesis of Peptide Beacon

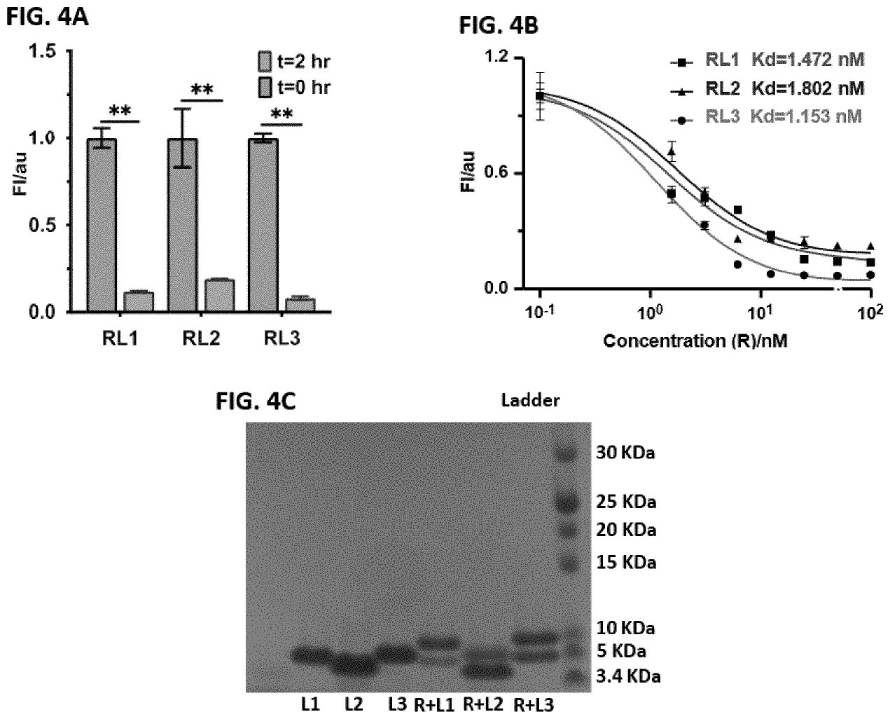

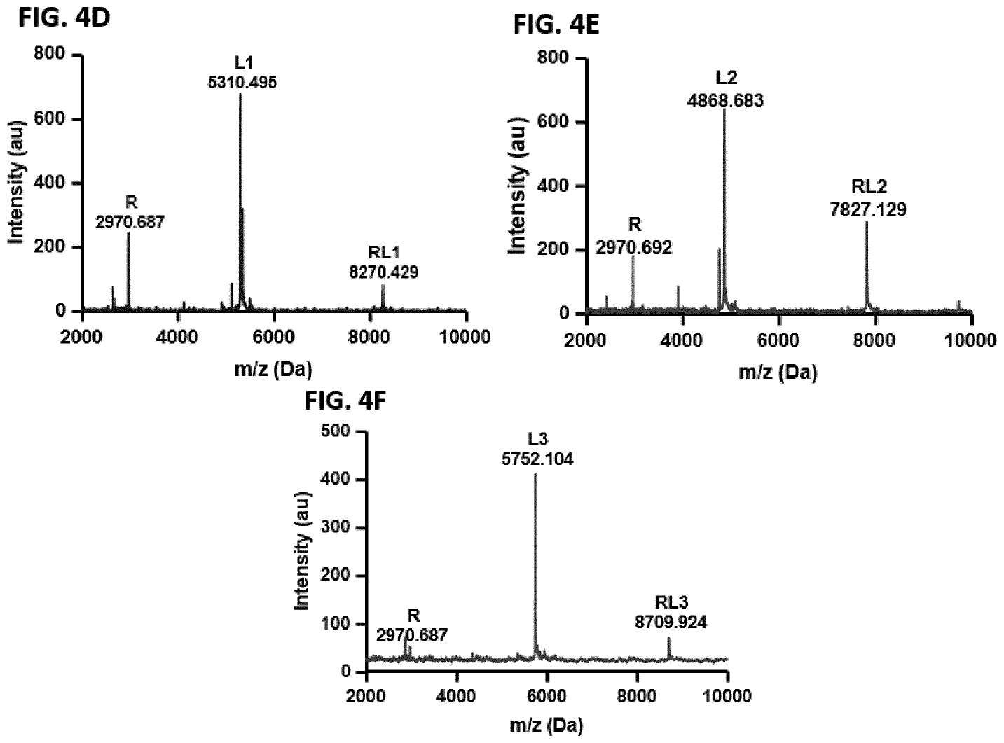

Referring now collectively to FIGS. 4A-4F, synthesis of peptide beacons, according to embodiments of the present disclosure, are demonstrated according to Example 1.

In FIG. 4A, the decrease in fluorescence intensity for each of peptide beacons RL1, RL2 and RL3, 2 hours after adding R subunits (50 nM in 1×PBS, pH 7.4) to L (50 nM in 1×PBS, pH 7.4), shows the conjugation between R and L forming peptide beacon. Residual fluorescence can be due to remaining unreacted L subunits.

In FIG. 4B, R units (400 nM to 100 pM in 1×PBS, pH 7.4) are titrated against 50 nM of L subunits in 1×PBS, pH 7.4 at 20-25° C. and the fluorescence intensities measured after 2 hours. Kd values of R subunits for L1, L2 and L3 subunits are represented, which show R subunits have affinity toward L subunits in preferential order: RL3>RL1>RL2. All the measurements were performed in triplicates (n=3) and the error bar represents the standard deviation of the data. Statistical analysis was performed using Unpaired t test in GraphPad software and the calculated P is represented as follows: **P<0.01.

In FIG. 4C, SDS-PAGE gel showing bands for L1, L2, L3, R+L1, R+L2 and R+L3. R+L1, R+L2 and R+L3 have two bands: one corresponds to L1, L2 or L3 subunit, second corresponds to the higher molecular weights near molecular weight of RL1, RL2 and RL3 conjugates.

In FIG. 4D, MALDI-TOF mass spectrum of R+L1 shows peaks corresponding to subunit R, subunit L1, and composite RL1. In FIG. 4E, MALDI-TOF mass spectrum of R+L2 shows peaks corresponding to subunit R, subunit L2, and composite RL2. In FIG. 4F, MALDI-TOF mass spectrum of R+L3 shows peaks corresponding to subunit R, subunit L3, and composite RL3. SDS-PAGE and mass spectroscopy were performed on the samplings taken from the R+L 2 hours after adding R subunits (˜50 nM in 1×PBS, pH 7.4) to L (˜100 nM in 1×PBS, pH 7.4).

### Example 2: Purifying Peptide Beacon Using HPLC

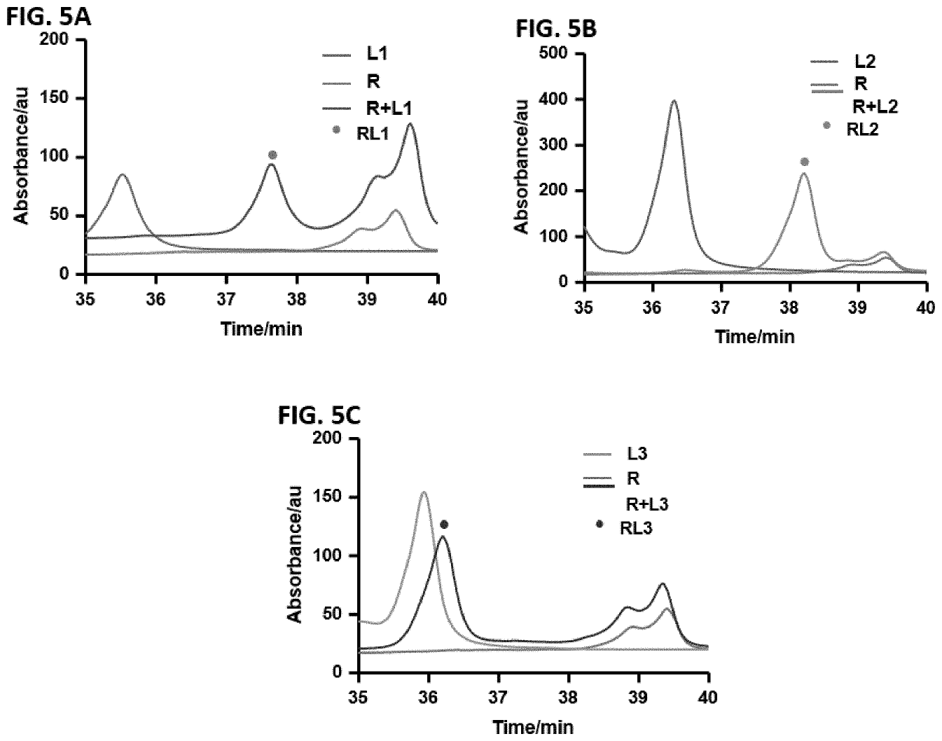

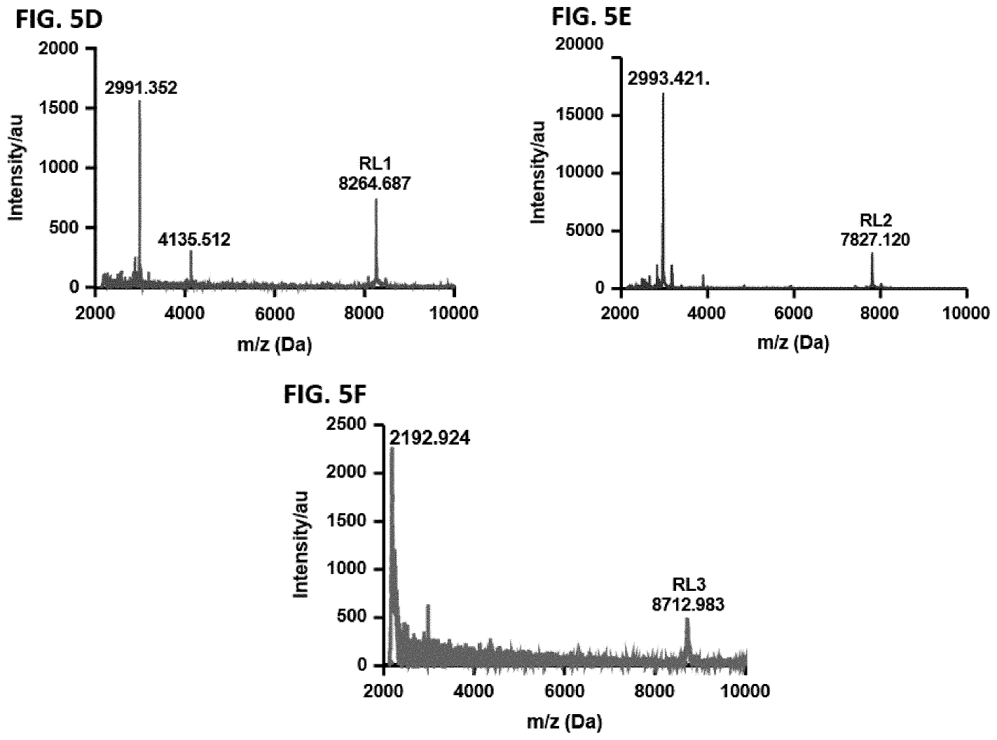

Referring now to FIGS. 5A-5F, purification of peptide beacons, according to embodiments of the present disclosure, are demonstrated according to Example 2.

In FIGS. 5A-5C, HPLC chromatograms of conjugates R+L1, R+L2 and R+L3 are plotted with HPLC chromatograms of subunits R, L1, L2 and L3. The chromatograms of conjugates R+L1 (FIG. 5A), R+L2 (FIG. 5B), and R+L3 (FIG. 5C) shows the appearance of new peaks having retention times of 37.645 min, 38.214 min and 36.210 min, respectively, which correspond to peptide beacons formed from conjugation between subunit R and subunit L. The material corresponding to new peaks were collected for a 1 minute duration centered around the retention time.

In FIGS. 5D-5F, MALDI-TOF mass spectrum of collected material in above HPLC analysis (FIGS. 5A-5C) of conjugates R+L1, R+L2 and R+L3, respectively. These spectra show peaks corresponding to the peptide beacons RL1 (FIG. 5D), RL2 (FIG. 5E), and RL3 (FIG. 5F), along with peaks near lower molecular weight, which may be due to the fractions of RL. Since the peaks in the HPLC analysis (FIGS. 5A-5C) were sharp and the material collected was from a narrow regime of retention period, the material collected were expected to have mostly RL1, RL2 and RL3. The measurements were performed on the samplings taken from the R+L, 2 hours after adding R subunits (˜50 nM in 1×PBS, pH 7.4) to L (˜100 nM in 1×PBS, pH 7.4). Purified RL1, RL2 and RL3 were used for detecting RBD of SARS-CoV-2 spike proteins and Influenza A-HA-H1.

### Example 3: Active Peptide Beacons with RBD of SARS-CoV-2 Spike Protein (Ab273065) and Influenza-HA (Ab217651) as Negative Control

Referring now collectively to FIGS. 6A-6E, detection of receptor binding domain (RBD) of the SARS-CoV-2 spike protein using peptide beacons, according to embodiments of the present disclosure, are demonstrated according to Example 3.

In FIG. 6A, a schematic shows the response of the peptide beacon to its target. The peptide beacon may comprise of a stem and loop. The stem may comprise a coiled-coil peptide and the loop may comprise a receptor for the intended target protein. A fluorophore-quencher pair may be attached to the two ends of the stem. When a target binds to the loop, the coiled-coil stem opens up moving the fluorophore away from the quencher, which increases the fluorescence yield of the system. The increase in the fluorescence yield of the system in response to the target can be exploited as a sensing mechanism.

FIG. 6B shows titration result for the target recombinant RBD of the SARS-CoV-2 spike protein (fM to μM in 1×PBS, pH 7.4) with three peptide beacons (5 nM in 1×PBS, pH 7.4). In FIG. 6C, titration of the target recombinant RBD of the SARS-CoV-2 spike protein (fM to μM in 1×PBS, pH 7.4) and Influenza A-HA-H1 (fM to μM in 1×PBS, pH 7.4) with RL1 (5 nM in 1×PBS, pH 7.4). FIG. 6D shows titration results for the target recombinant RBD of the SARS-CoV-2 spike protein (fM to μM in 1×PBS, pH 7.4) and Influenza A-HA-H1 (fM to μM in 1×PBS, pH 7.4) with RL2 (5 nM in 1×PBS, pH 7.4). FIG. 6E shows titration results for the target recombinant RBD of the SARS-CoV-2 spike protein (fM to μM in 1×PBS, pH 7.4) and Influenza A-HA-H1 (fM to μM in 1×PBS, pH 7.4) with RL3 (5 nM in 1×PBS, pH 7.4). For this example, all the measurements were performed in triplicates (n=3) at 20-25° C. and the error bar represents the standard deviation. Kd values shows the sensitivity of peptide beacons in the order RL2>RL1>RL3 and the specificity towards RBD is in the order RL3>RL1>RL2.

Attached hereto as Appendix A, which is herein fully incorporated by reference, is a draft pre-publication journal submission related to the disclosed embodiments of the present invention.

While certain embodiments of the present disclosure are discussed herein, many other implementations will occur to one of ordinary skill in the art and are all within the scope of the invention. Each of the various embodiments described above may be combined with other described embodiments in order to provide multiple features.

Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. A peptide probe for detection of a target protein, the peptide probe comprising:
a stem having a first end and a second end, a fluorophore attached to the first end and a quencher attached to the second end; and
a loop proximately located between the first and second ends, the loop comprising a receptor sequence capable of binding with the intended target protein;
wherein the first and second ends are configured to form a coiled-coil structure when the receptor sequence is unbound from the intended target protein; and
wherein the first and second ends are configured to form an open-coil structure when the receptor sequence is bound with the intended target protein.

2. The peptide probe of claim 1, wherein the peptide probe is in a low-fluorescence state in the coiled-coil structure and a high-fluorescence state in the open-coil structure.

3. The peptide probe of claim 2, wherein the low-fluorescence state occurs when a distance between the fluorophore and the quencher is about the Förster distance or less, and
wherein the high-fluorescence state occurs when a distance between fluorophore and the quencher is greater than the Förster distance.

4. The peptide probe of claim 1, wherein the receptor sequence comprises SEQID No. 8.

5. The peptide probe of claim 1, wherein the peptide probe comprises a peptide sequence comprising one of SEQID No. 1, SEQID No. 2, SEQID No. 3, SEQID No. 4, SEQID No. 5, SEQID No. 6, and SEQID No. 7.

6. The peptide probe of claim 1, wherein one of the first end and the second end of the stem comprises a peptide sequence comprising SEQID No. 1.

7. The peptide probe of claim 6, wherein the first end comprises SEQID No. 1, and the second end comprises at least a portion of a peptide sequence comprising one of SEQID No. 2, SEQID No. 3, and SEQID No. 4.

8. The peptide probe of claim 1, wherein the spike protein receptor binding domain is associated with a coronavirus.

9. The peptide probe of claim 1, wherein the peptide probe is configured to detect a SARS-CoV-2 spike protein with a LoD of about 50-60 pM.

10. The peptide probe of claim 1, wherein the peptide probe is configured to produce at least a detectable fluorescence signal when the peptide probe transitions from the coiled-coil structure to the open-coil structure; and
wherein the peptide probe transitions from the coiled-coil structure to the open-coil structure within a turn-around time of less than 10 minutes.

11. The peptide probe of claim 1, wherein the peptide probe is integrated with an on-chip optical sensor to construct a point-of care antigen test platform for at least one of: a virus, a coronavirus, and SARS-CoV-2.

12. The peptide probe of claim 1, wherein the target protein is a polypeptide.

13. The peptide probe of claim 1, wherein the target protein is a spike protein receptor binding domain.

14. The peptide probe of claim 1, wherein the target protein is part of a viral envelope.

15. The peptide probe of claim 1, wherein the target protein is part of a coronavirus.

16. The peptide probe of claim 1, wherein the target protein is part of a SARS-CoV-2 virus.

17. The peptide probe of claim 1, wherein the peptide probe is able to detect a SARS-CoV-2 spike protein with a LoD of about 50-60 pM.

18. A system for the detection of an intended target protein virus, the system comprising:
a peptide probe comprising:
a stem section having a first end and a second end;
a fluorophore-quencher pair attached to the first and second ends; and
a loop section proximately located between the first end and the second end, the loop comprising a receptor sequence capable of binding with the intended target protein;
wherein the peptide probe is configured to have a coiled-coil structure in the absence of the receptor sequence binding with the intended target protein;
wherein the peptide probe is configured to have an open-coil structure in the presence of the receptor sequence binding with the intended target protein; and
wherein the open-coil structure generates a detectable fluorescence signal.

19. The system of claim 18, further comprising a sample selected from the group consisting of blood, saliva, urine, nasal fluid, nasopharyngeal fluid, oropharyngeal fluid, condensed breath, or combination thereof.

20. A method for selecting sequences of a peptide beacon comprising:
obtaining one or more coiled coil sequences;
docking the one or more coiled coil sequences against a target of interest;
predicting a binding strength score based on the docking step;
testing the one or more coiled coil sequences against the target using a degradation assay; and
determining, according to the docking and the testing, a first sequences of the one or more coiled coil sequences to use as a right arm of the peptide beacon and a second sequence of the one or more coiled coil sequences to use as a left arm of the peptide beacon.

21. A method for designing a binding loop of a peptide beacon comprising:
supplying a distogram of a right arm and a left arm of a coiled-coil peptide, each of the right arm and the left arm comprising a coiled-coil sequence, to a generative model configured to predict an initial random sequence; and
iteratively optimizing the initial random sequence using loss based on sequence constraints.

22. The method of claim 21, wherein iteratively optimizing the initial random sequence comprises using a distogram prediction model to predict a distogram of a complete peptide beacon, and
wherein the complete peptide beacon comprises the right arm, the left arm, and the initial random sequence.

23. The method of claim 21, wherein iteratively optimizing the initial random sequence comprises using loss further based on one of: coiled motif loss and generative loss.

24. The method of claim 21, wherein the sequence constraints are selected based on a target percent similarity to a known binding partner to the target.
